(19) United States
(12) Patent Application Publication
(10) Pub. No.: US 2015/0366571 A1
(43) Pub. Date: Dec. 24, 2015

(54) IMAGE-BASED COMPUTER-AIDED SAFE STONE EXTRACTION ADVISOR

(71) Applicant: GYRUS ACMI, INC. (d.b.a. Olympus Surgical Technologies America), Southborough, MA (US)

(72) Inventors: Adi Navve, Kfar Saba (IL); Shai Finkman, Haifa (IL)

(21) Appl. No.: 14/313,308

(22) Filed: Jun. 24, 2014

(51) Int. Cl. A61B 17/221; A61B 19/00

(52) U.S. Cl. CPC A61B 17/221; A61B 19/5212; A61B 2019/463; A61B 2017/22038

(57) ABSTRACT

An endoscopist is informed whether a stone or fragment can be extracted through a lumen without injury by detecting a minimum lumen size, detecting a maximum stone size, comparing the minimum lumen size with the maximum stone size, determining that the maximum target stone size is less than the minimum lumen size, and removing the target stone through the lumen.

### BACKGROUND OF THE INVENTION

1. Field of the Invention

This invention relates to removal of calculi from the body. More particularly, this invention relates to extraction of urinary calculi.

2. Description of the Related Art

Nowadays, lithotripsy for urinary stones can be carried out endoscopically, with or without extracorporeal shockwave or intracorporeal lithotripsy. intracorporeal lithotripsy may be conducted by flexible or rigid ureteroscopy or percutaneous nephrolithotomy. Intracorporeal lithotripsy is typically accomplished using laser energy. However, other technologies such as ballistic lithotripsy, ultrasonic lithotripsy and electrohydraulic lithotripsy are applied by instrumentation of the urinary tract.

In any case, in order to avoid injury to tissue, it is important to determine whether the urinary stone or a fragment thereof can be extracted using a retrieval device or pass through the urinary vessels spontaneously. If not, further comminution of the stone, e.g., by lithotripsy, may be necessary in order to assure successful removal.

For example, stone size and location are important determinants to predict spontaneous stone passage. The ureter is the structure having the smallest diameter of the urinary tract and is the urinary vessel most likely to be obstructed by a stone. Stones (or fragments) less than about 5 mm in diameter are usually likely to pass spontaneously. However, the likelihood of spontaneous stone passage through the ureter depends on not only the stone size, but also the differences of vessel size among individuals and ages, and whether there is a stricture in the vessel. In addition, the likelihood of stone retrieval through a channel of an instrument depends on the channel size of an endoscope and whether an access sheath is utilized for retrieving a stone.

U.S. Patent Application Publication No. 2010/0092054 proposes configuration of an endoscope to include an element, such as a standard having a known dimension. The endoscope is introduced into a subject so that the standard is adjacent the item to be examined. Based on known parameters of the standard, the size of the area being examined can be estimated.

U.S. Patent Application Publication No. 2004/0242961 discloses a measurement system for indirectly measuring dimensions of a defect in a target area. The measurement system comprises illuminating optics for illuminating the target area, a laser pattern generator with a laser source and a pattern generating assembly for producing a reference laser pattern having at least one reference dimension that is essentially invariant within a range of working distances. The reference laser pattern is projected onto the target area, and an imaging system obtains an image of the target area together with the reference pattern.

U.S. Pat. No. 8,607,634 discloses using a transducer to send an ultrasound pulse toward a stone and to receive ultrasound reflections from the stone regarding the measurement of the size of a stone. The recorded time between a pulse that is reflected from the proximal surface and a pulse that is reflected from the distal surface of the stone or from a surface supporting the stone is used to calculate the stone size. The size of the stone is a function of the time between the two pulses and the speed of sound through the stone (or through the surrounding fluid if the second pulse was reflected by the surface supporting the stone).

### SUMMARY OF THE INVENTION

According to embodiments of the invention, methods and systems are provided to inform an endoscopist whether a stone or fragment can be extracted without injury.

There is provided according to embodiments of the invention a method for removal of a target stone in a body through a lumen. The target stone (sometimes referred to herein as a “calculus”) may include a stone, a stone fragment, or a group of stone fragments. The method is carried out by detecting a minimum lumen size, detecting a maximum target stone size of the target stone, comparing the minimum lumen size and the maximum stone size, determining that the maximum target stone size is less than the minimum lumen size, and removing the target stone through the lumen.

In one aspect of the invention, the detecting steps may include inserting an imaging device into the body through the lumen of a body, acquiring at least one first image of the lumen through the imaging device, acquiring at least one second image of the target stone through the imaging device, and determining the minimum lumen size and the maximum stone size by processing the images.

In another aspect of the invention, the detecting steps may include inserting an imaging device having a channel as the lumen to retrieve the target stone and having a memory in which the minimum lumen size is stored, and comparing the minimum lumen size with the maximum stone size by processing the images.

In a further aspect of the invention, the detecting steps may include inserting an imaging device through a body lumen, the imaging device having a channel lumen to retrieve the target stone and a memory in which the minimum channel lumen size is stored, acquiring at least one first image of the body lumen through the imaging device, acquiring at least one second image of the target stone through the imaging device, comparing the minimum body lumen size with the minimum channel lumen size, and determining the minimum lumen size and the maximum stone size by processing the images.

In yet another aspect of the invention, removing the target stone includes inserting a retrieval device into the lumen, and removing the target stone by the retrieval device after determining the minimum lumen size and the maximum stone size.

In still another aspect of the invention, removing the target stone includes inserting a lithotripsy device to reduce the maximum size of the target stone, and removing the target stone, after reduction, by the retrieval device after determining the minimum lumen size and the maximum stone size.

In another aspect of the invention determining the minimum lumen size and the maximum stone size include acquiring a reference image from a reference object, wherein the reference object has a first known dimension and the lumen or the target stone has a second dimension, determining a first number of pixels in the reference image that are occupied by the first known dimension, determining a second number of pixels in the reference image that are occupied by the second dimension, and calculating a size of the lumen, a size of the target stone, or both, by comparing the first number of pixels with the second number of pixels.

In yet another aspect of the invention the reference object includes a safety guidewire that extends through the vessel.

In still another aspect of the invention, the retrieval device also includes a reference object to detect at least the size of the body lumen or target stone.

In an additional aspect of the invention, the retrieval device also includes a shaft to be inserted into the lumen, and the reference object is the shaft.

In another aspect of the invention, the retrieval device also includes a wire to retrieve the target stone, and the reference object is the wire.

### BRIEF DESCRIPTION OF THE SEVERAL VIEWS OF THE DRAWINGS

For a better understanding of the present invention, reference is made to the detailed description of the invention, by way of example, which is to be read in conjunction with the following drawings, wherein like elements are given like reference numerals, and wherein:

FIG. 1 is a pictorial schematic diagram of a system in accordance with an embodiment of the invention;

FIG. 2 is a composite drawing illustrating techniques of endoscopy suitable for use with the system shown in FIG. 1 in accordance with alternate embodiments of the invention;

FIG. 3 is a schematic diagram illustrating the distal end of the endoscope shown in FIG. 1 in accordance with an embodiment of the invention;

FIG. 4 is a schematic diagram in which an endoscope is in a mode of operation for making measurements in accordance with an embodiment of the invention;

FIG. 5 is a schematic image of a urinary vessel acquired by an endoscope, illustrating the measurement of a urinary vessel in accordance with an embodiment of the invention;

FIG. 6 is an image of a calculus acquired by an endoscope in accordance with an embodiment of the invention;

FIG. 7 is a flow chart of a method of image-based computer-aided safe stone extraction in accordance with an embodiment of the invention;

FIG. 8 is a schematic diagram illustrating the execution by the endoscope shown in FIG. 1 of a stone retrieval operation in accordance with an alternate embodiment of the invention;

FIG. 9 is a flow-chart of a method of extraction of a stone through the channel of an endoscope in accordance with an alternate embodiment of the invention;

FIG. 10 is a flow-chart of a method of method of extraction of a stone in accordance with an alternate embodiment of the invention; and

FIG. 11 is a schematic diagram illustrating the execution by the endoscope shown in FIG. 1 of a stone retrieval operation in accordance with an alternate embodiment of the invention.

### DETAILED DESCRIPTION OF THE INVENTION

In the following description, numerous specific details are set forth in order to provide a thorough understanding of the various principles of the present invention. It will be apparent to one skilled in the art, however, that not all these details are necessarily always needed for practicing the present invention. In this instance, well-known circuits, control logic, and the details of computer program instructions for conventional algorithms and processes have not been shown in detail in order not to obscure the general concepts unnecessarily.

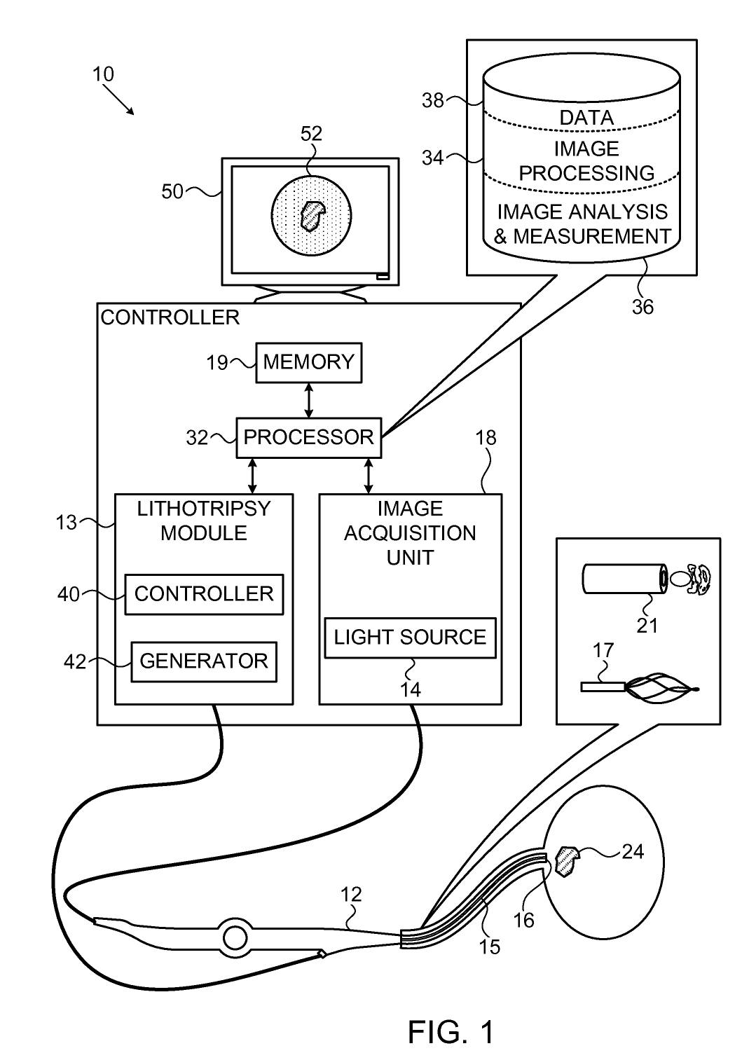

Turning now to the drawings, Reference is initially made to FIG. 1, which is a pictorial schematic diagram of a system 10, in accordance with an embodiment of the invention. A conventional endoscope 12 is adapted with a retrieval device 17 for intracorporeal retrieval of urinary calculi. A helical stone basket is shown in FIG. 1 as the retrieval device 17; however, this is by way of example and not of limitation. The endoscope 12 may be equipped for any form of intracorporeal stone retrieval including forceps, basket device, balloon catheter and combinations thereof.

The endoscope 12 may also be equipped with a lithotripsy probe 21, such as the exemplary electrohydraulic lithotripsy probe shown in FIG. 1. The lithotripsy probe 21 can involve any form of intracorporeal lithotripsy known in the art, including laser lithotripsy, electrohydraulic lithotripsy, pneumatic lithotripsy, ultrasonic lithotripsy, and combinations thereof.

The retrieval device 17 and the lithotripsy probe 21 are operated via a working channel 15 in the endoscope 12.

The principles of the invention are applicable to endoscopic procedures in the human or animal body that do not include lithotripsy, such as removal of foreign objects. The endoscope 12 can be a ureteroscope or a nephroscope adapted for percutaneous entry to the renal pelvis.

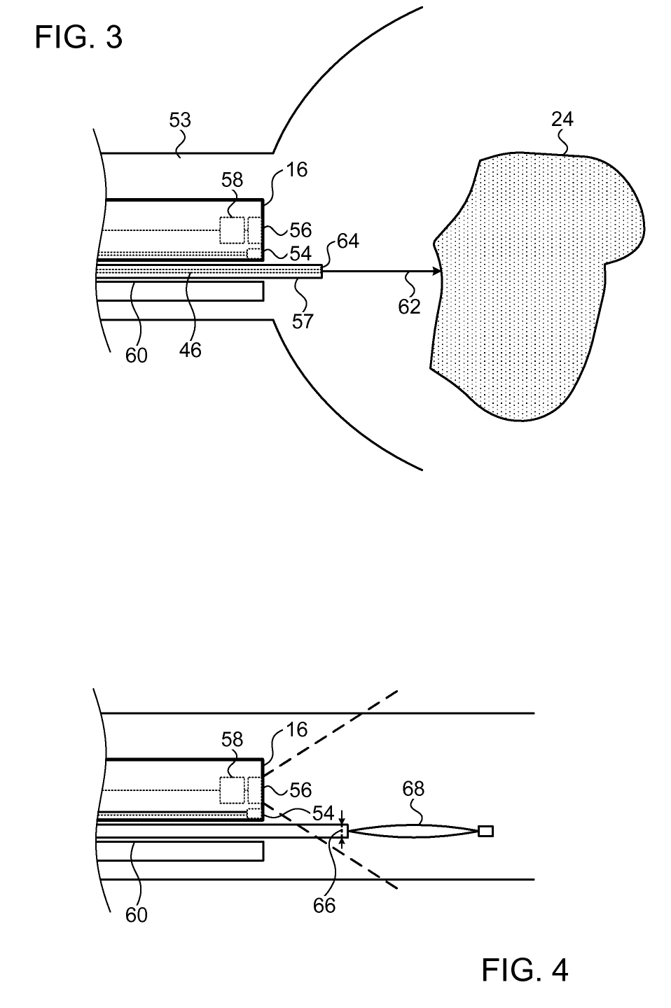

Energy produced by a lithotripsy module 13 to a laser optical probe 57, which is projected through the working channel 15 of the endoscope 12, the laser optical probe comprising fiberoptics and an optical lens (not shown) for transmitting laser energy from a generator 42 to calculus 24. The endoscope 12 includes a lens system and semi-conducting imaging array at distal end 16 for returning reflected light to an image acquisition unit 18. A light source 14 may emit light at one or more wavelengths for illumination of the calculus 24 and the body lumen. The optics are described below in further detail in the discussion of FIG. 3.

The image acquisition unit 18 can be realized as the device described in U.S. Pat. No. 8,659,646, which is herein incorporated by reference.

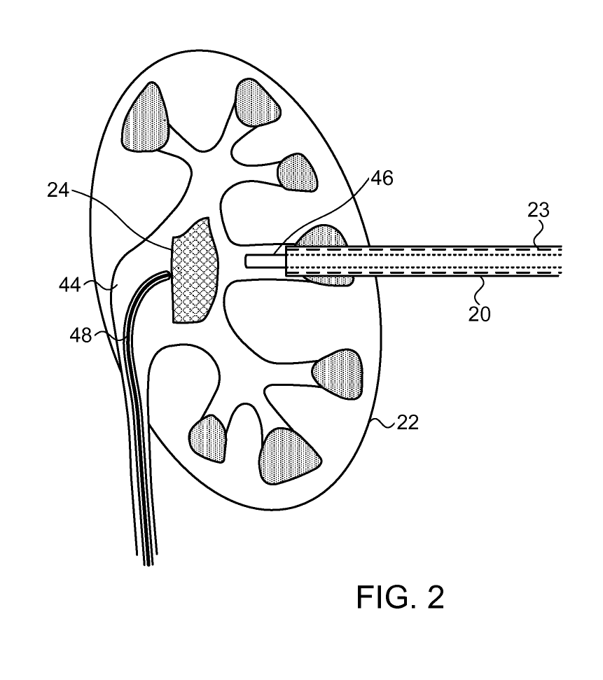

Reference is now made to FIG. 2, which is a composite schematic drawing illustrating techniques of treating urinary calculi that are suitable for use with the system 10 (FIG. 1), in accordance with embodiments of the invention. In some embodiments of the system 10, a nephroscope 20 functions as an endoscope and is connected to the image acquisition unit 18. The nephroscope 20 enters kidney 22 percutaneously to visualize calculus 24 located in renal pelvis 44. The nephroscope 20 has a hollow channel 23 through which the retrieval device 17 (FIG. 1) and an optical fiber 46 can be inserted and placed in proximity with the calculus 24. Alternatively, a ureteroscope 48 can be passed in a retrograde direction through the urinary tract to approach the calculus 24. The nephroscope 20 and ureteroscope 48 can incorporate the various intracorporeal lithotripsy technologies noted above. Image analysis may be directed to fragments of the calculus 24 before and after lithotriptic or mechanical comminution.

Reverting to FIG. 1, the image acquisition unit 18 provides image data to a processor 32. The processor 32 typically comprises a general purpose or embedded computer processor, which is provided with a memory 19, and programmed with suitable software for carrying out the functions described hereinbelow. Thus, although the processor 32 is shown as comprising a number of separate functional blocks, these blocks are not necessarily separate physical entities, but rather represent different computing tasks or data objects stored in a memory that is accessible to the processor. These tasks may be carried out in software running on a single processor, or on multiple processors. The software may be embodied on any of a variety of known non-transitory media for use with a computer system, such as a diskette, or hard drive, or CD-ROM. The code may be distributed on such media, or may be distributed to the processor 32 from the memory or storage of another computer system (not shown) over a network. Alternatively or additionally, the processor 32 may comprise a digital signal processor or hard-wired logic.

During insertion of the endoscope 12, while the operator is illuminating the body lumen, the image acquisition unit 18 may also acquire an image of the body lumen. The processor 32 is programmed to execute image-processing routines 34 to measure the size of the body lumen, and detects the minimum size of the body lumen (e.g., a body vessel) using analysis programs 36, as described in further detail below. In addition, the image acquisition unit 18 acquires an image of the target stone, and the processor 32 is programmed to execute image-processing routines to measure the target stone size and detect the maximum target stone size. An optional database 38 may accumulate experience using the analysis developed by the image processing routines 34. Reference to the database 38 may be useful for refining predictions of the image processing routines 34. The processor 32 programmed to execute image-processing routines 34 to determine that the maximum target stone size is less than the minimum lumen size by comparing the maximum target stone size with the minimum body lumen size.

Reference is now made to FIG. 3, which is a schematic diagram illustrating the distal end 16 of endoscope 12 (FIG. 1), in accordance with an embodiment of the invention. The distal end 16 is assumed to be lie within a body lumen or urinary vessel lumen 53 in proximity to calculus 24. An illumination lens 54 is able to radiate visible light, typically white light, under control of the image acquisition unit 18 (FIG. 1). Returning light from an object illuminated by the illumination lens 54 is focused by a lens system 56 onto a semiconducting imaging array 58, which is also controlled by the image acquisition unit 18, and which enables capture of an image of the illuminated object. The laser optical probe 57 traverses working channel 60 and is configured to be able to transmit a laser beam produced by generator 42 in the lithotripsy module 13 through the optical fiber 46 and along a path 62 extending from distal end 64 to decrease the size of the calculus 24. The laser beam conveys sufficient energy to break or fracture calculus 24. During laser lithotripsy, a fiber tip at distal end 64 is placed in contact with the stone's surface or at in close proximity to the stone, typically within 1 mm. By calculating the relation between the size of the tip in the image with the size of the stone or stone fragment in the image, based on the absolute size of the tip, the size of the stone or stone fragment can be calculated.

Calculation of stone size may also be based on detection of the laser aiming beam. During laser lithotripsy an aiming beam, having a red or green color, is transmitted through the fiber along together with an ablating laser beam, which is invisible to the human eye. The visible beam indicates the location of the target. The beam diameter seen on the surface of the stone is determined by the known size of the fiber used. The stone and fragment sizes may be calculated with reference to the beam diameter. Further details of this technique are presented in the above-noted application Ser. No. 14/274,726.

As noted above, the apparatus associated with the laser beam, while commonly employed, is optional.

Reference is now made to FIGS. 4, 5 and 6, which are schematic diagrams, in which an endoscope is in a mode of operation for making measurements, in accordance with an embodiment of the invention. An image of a reference object 66 is acquired by the imaging array 58. The reference object 66 may be, e.g., a shaft or basket wire of a basket device 68 (shown unexpanded in FIG. 4).

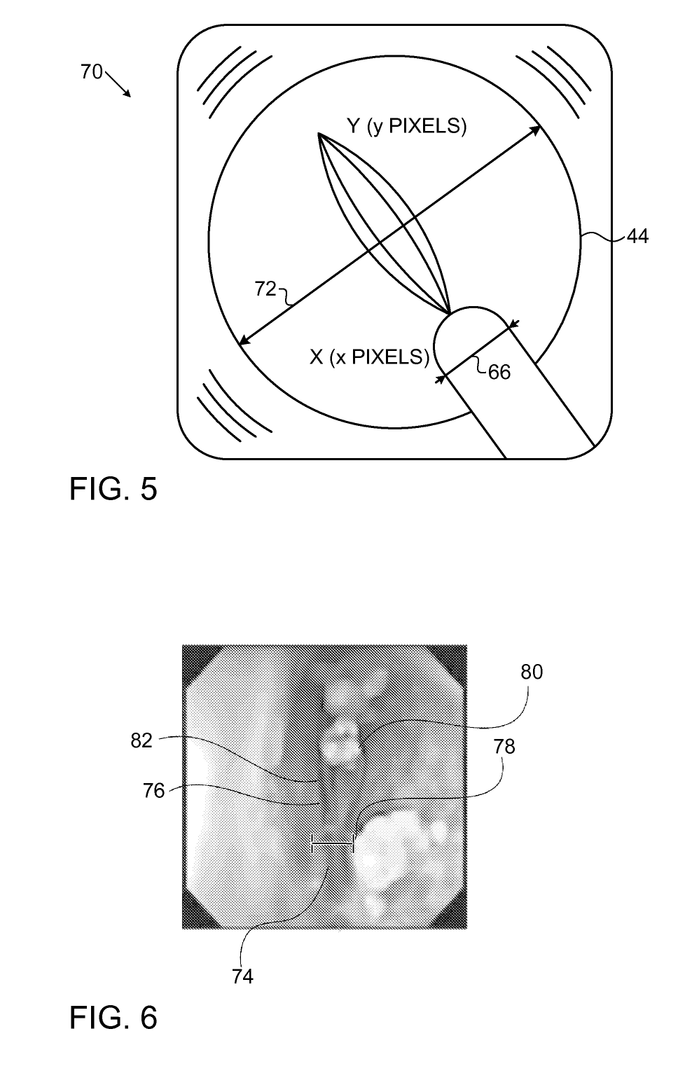

FIG. 5 is a schematic diagram of an image 70 a urinary vessel lumen 44 acquired by the imaging array 58 in the arrangement of FIG. 4, in accordance with an embodiment of the invention. The image processing routines 34 (FIG. 1) measures the size of urinary vessel lumen 44 (Y) by comparing the number of y pixels with the number of x pixels, (x and y can be calculated as reference X has a known size). In case the calculus 24 is placed in the image (not shown in FIG. 5), the image processing routines 34 (FIG. 1) measure the size of the target stone 24 in the same way. In the course of the introduction of the endoscope, other frames may be acquired, which in general show different segments of the urinary vessel lumen 44. Such frames may be analyzed individually. The image processing routines 34 detects the maximum target stone size and the minimum lumen size, then the image processing routines 34 determine that the maximum target stone size is less than the minimum lumen size.

As noted above, many algorithms are known for determining the size of an object through an endoscope. Objects of known size can be used as scaling elements: a laser's aiming beam, a safety guidewire of known dimensions running through the ureter into the kidney; the laser fiber's tip; a lithotripter tip. The most commonly used guidewires for ureteral access are 0.035 in or 0.038 in. (see Waingankar, N., Okeke, Z., & Smith, A. D. (2013). Guidewires and Angled Catheters. In M. Monga (Ed.), Ureteroscopy Indications, Instrumentation & Technique (pp. 127-136)) Therefore, the system could assume that the guidewire is one of the standard guidewires, and use an average size of 0.0365 in. In case that the guidewire projected through the channel is visualized, the channel lumen size is recognized at least as well as the size of guidewire. That is to say, the minimum channel lumen size can be detected.

An orbiculate edge of the vessel can be extracted by an image processing, for example, by binarization of brightness of the vessel wall illuminated by the illumination lens 54 (FIG. 4). The processor is configured to calculate the number of pixels edge-to-edge (diameter 72) and on the reference object 66. The diameter of the tip is stored in the memory. The processor can determine the minimum size of a diameter of the edge by comparing the number of pixels edge-to-edge with the reference.

Alternatively, An ultrasonic probe can be extended through the working channel 60, and the diameter of the vessel can be measured during the insertion of the endoscope. Based on the data obtained by ultrasonic probe, the minimum diameter can be determined by the processor. Image processing can be used to detect the target stone. Based on the color of the stone, a contour can be recognized. The diameter of the contour can be measured and the maximum diameter can be detected based on the contour and the reference.

Alternatively, the diameter of the urinary vessel (or instrument), or the size of the stone, may be obtained by combining: 1) image processing methods known in the art as ‘Shape from Motion’ algorithms, which utilize a sequence of consecutive images captured while the camera is moving, and 2) information on the movement of the camera as obtained by motion or location sensors incorporated in the endoscope. Alternatively, the diameter of the urinary vessel 44 may be obtained using the method of the above-referenced U.S. Patent Application Publication No. 2010/0092054 or U.S. Patent Application Publication No. 2004/0242961, which is herein incorporated by reference.

Reference is now made to FIG. 6, which is an image of a calculus acquired by an endoscope, in accordance with an embodiment of the invention. A shaft 74 basket device 76 is marked by bracketed line 78. The basket has been expanded to capture a stone fragment 80. Either the shaft 74 or basket wires 82 can serve as a reference object for measuring the fragment 80 or the body lumen. Often only the wires 82 are visible.

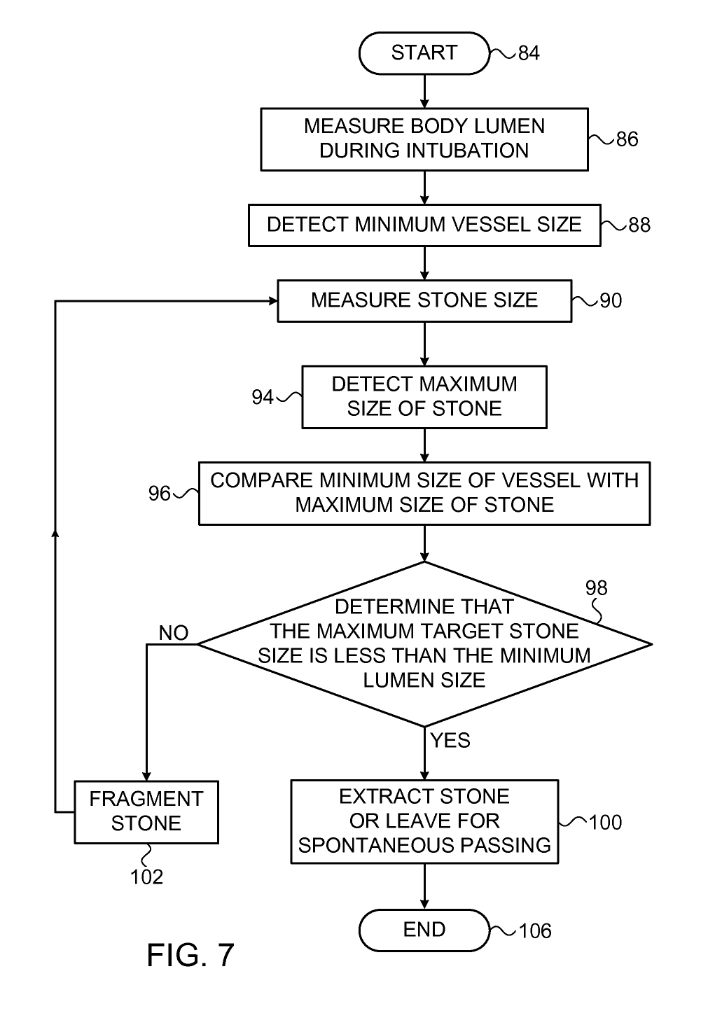

Reference is now made to FIG. 7, which is a flow-chart of a method of image-based computer-aided safe stone extraction, in accordance with an embodiment of the invention. The process steps are shown in a particular linear sequence in FIG. 7 for clarity of presentation. However, it will be evident that many of them can be performed in parallel, asynchronously, or in different orders. Those skilled in the art will also appreciate that a process could alternatively be represented as a number of interrelated states or events, e.g., in a state diagram. Moreover, not all illustrated process steps may be required to implement the process.

The method shown in FIG. 7 may be performed automatically using the facilities of the system 10 (FIG. 1).

The procedure begins at initial step 84. A subject is intubated with an endoscope, typically a ureteroscope or a nephroscope as described above. One way of performing initial step 84 and corresponding intubation steps in the other embodiments hereof is the use of a ureteral access sheath of the type described in U.S. Pat. Nos. 8,535,293 and 8,235,968, both assigned to Gyrus ACMI, Inc., and herein incorporated by reference. One sheath of this sort is available from Gyrus ACMI as the UroPass® ureteral access sheath and comprises a wide funnel-shaped hub that guides ureteroscopes and retrieval devices into the sheath without the need for direct visualization or orientation.

Next, at a step 86 a series of images of the vessel is captured and the lumen size is measured as the endoscope is advanced toward the target stone.

At step 88 the minimum vessel lumen size is detected respectively on the images that were acquired in step 86 using one of the methods described above.

At step 90, the endoscope is placed into contact with or in proximity with a target stone in a vessel or body lumen. The target stone can be a stone, a stone fragment, or a group of stone fragments. The endoscope is provided with optical imaging capabilities and an optional energy delivery system as noted above. An optical image of the target stone is acquired and the target stone size is measured.

Next, at step 94, the image obtained in step 90 is analyzed to detect the maximum size of the target stone, using one of the techniques described above.

Next, at step 96, a comparison is made between the maximum size of the target stone and the minimum size of the urinary vessel lumen. Typically, the target stone is irregular, and its maximum dimension may initially be compared to the minimum diameter of the urinary vessel lumen.

Next, at decision step 98, it is determined if, based on the comparison of step 96, the target stone is extractable or can be passed spontaneously. If the maximum diameter of the target stone is less than the minimum diameter of the urinary vessel, then it is concluded that this is the case. Otherwise, it is concluded that the target stone cannot presently be extracted or passed.

If the determination is affirmative then control proceeds to step 100. The target stone is extracted or is allowed to remain in order to pass spontaneously. Extraction may be performed using any known technique. For example, a stone or group of stones may be grasped in a basket extraction device. In the case of smaller fragments, i.e., those less than 0.5 cm, a judgment may be made by the operator to extract them, or to allow them to be expelled spontaneously. The procedure then ends at final step 106.

If the determination at decision step 98 is negative then control proceeds to step 102. Fragmentation of the target stone is performed using known lithotriptic or mechanical techniques, including the intracorporeal lithotryptic methods described in commonly assigned copending U.S. application Ser. No. 14/274,726, entitled “Computer Aided Image-Based Enhanced Intracorporeal Lithotripsy”, which is herein incorporated by reference.

After performing step 102 control returns to step 90 to iterate on other remnants of the stone. As noted above, small fragments may be left in place or extracted with a retrieval device, according to the judgment of the operator.

### First Alternate Embodiment

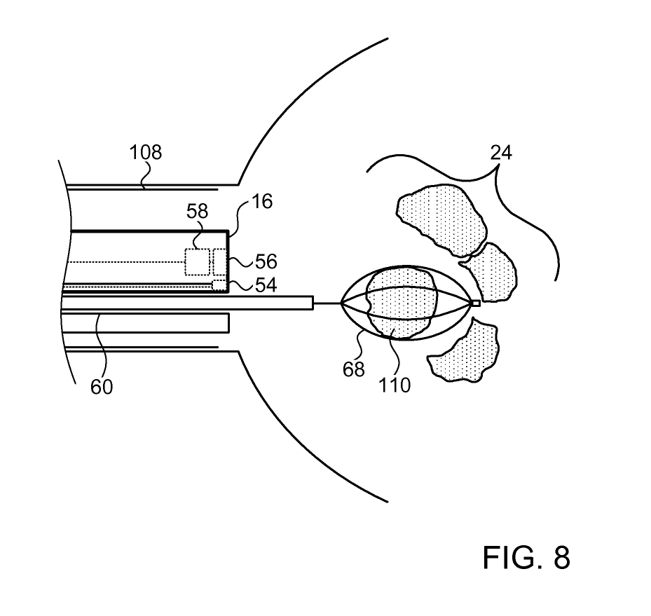

Reference is now made to FIG. 8, which is a schematic diagram illustrating the execution of a stone retrieval operation by the endoscope 12 subsequent to comminution of the calculus 24, in accordance with an alternate embodiment of the invention. An access sheath 108 may be employed to assist in placing the endoscope. The basket device 68 is configured to be inserted through the working channel 60 to retrieve the calculus 24 through the working channel 60, The calculus 24 is now fragmented subsequent to an activation of the laser beam and the lithotripsy module 13 (FIG. 1). One fragment 110 has been entrapped by the basket device 68 Images of the basket device 68 may be acquired by the imaging array 58.

In this embodiment, the image acquisition unit 18 (FIG. 1) can detect the characteristics of the particular type of endoscope being employed as the endoscope 12, and data regarding its channel size is stored in the memory 19. The processor 32 consults the memory 19 to recognize the size of the working channel 60 and to compare it with the size of the calculus 24.

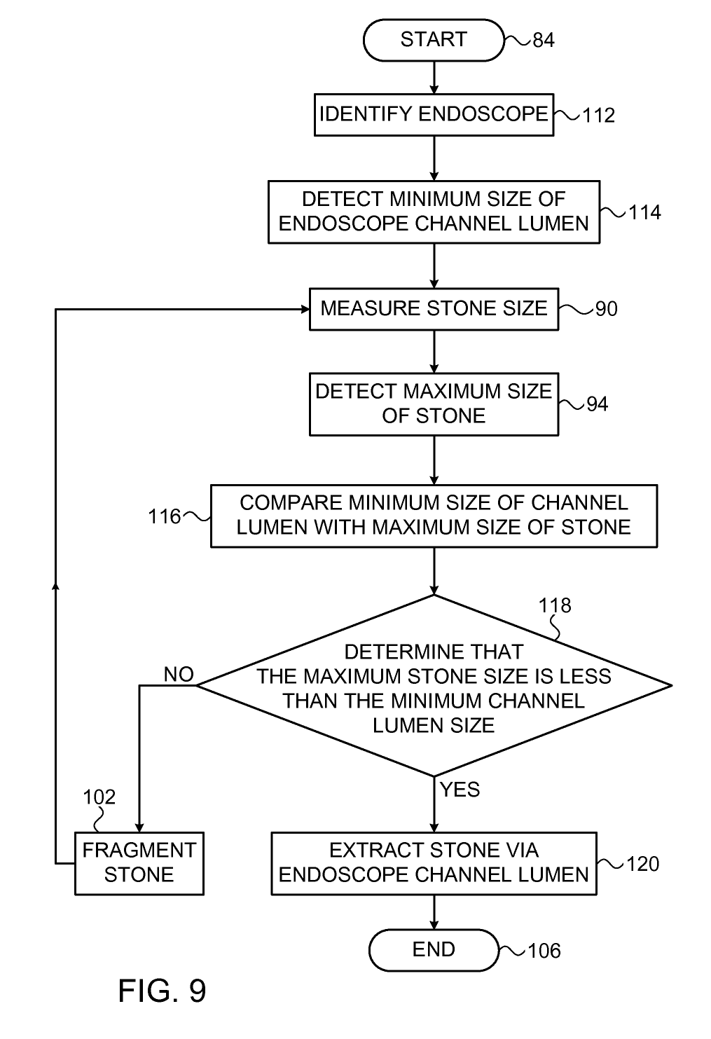

Reference is now made to FIG. 9, which is a flow-chart of a method of extraction of a stone through the channel of an endoscope, in accordance with an alternate embodiment of the invention. The general conditions described with regard to the discussion of FIG. 7 are applicable to this embodiment. The descriptions of steps that are identical to those of FIG. 7 are not repeated in the interest of brevity.

After performing initial step 84, the characteristics of the endoscope being used are identified in step 112.

Next, at step 114 the minimum size of the endoscope channel is made known to the system processor. This may be accomplished by assuming an average size as described above. Alternatively, a signature or identifier of the endoscope may be readable, and the required dimension may then be recovered from a database stored in memory. Further alternatively, the dimension may be obtained from the manufacturer's specifications and entered manually into the system by the operator.

After performing steps 90, 94 as described above, next, at step 116, a comparison is made between the maximum size of the target stone (calculus) and the minimum size of the endoscope channel lumen.

Next, at decision step 118, based on the comparison of step 116, it is determined if the calculus is extractable, i.e., whether the maximum size of the calculus is less than the minimum channel lumen size.

If the determination is affirmative then control proceeds to step 120. The target stone is extracted via the endoscope channel using any suitable retrieval device, such as a wire basket as described above. As in the previous embodiment, for smaller fragments, i.e., those less than 0.5 cm, a judgment may be made by the operator to extract them, or to allow them to be expelled spontaneously. The procedure terminates at final step 106.

If the determination at decision step 118 is negative then control proceeds to step 102 as described above.

### Second Alternate Embodiment

Referring again to FIG. 3, in this embodiment it is possible to remove the calculus 24 either through the working channel 60 of the endoscope or through the vessel lumen 53 without bringing the calculus 24 through the interior of the endoscope. In this embodiment, the image acquisition unit is aware of the minimum dimension of the working channel 60 of the endoscope, as described in step 114 (FIG. 9). Moreover, the minimum size of the vessel lumen is determined as described in step 92 (FIG. 7).

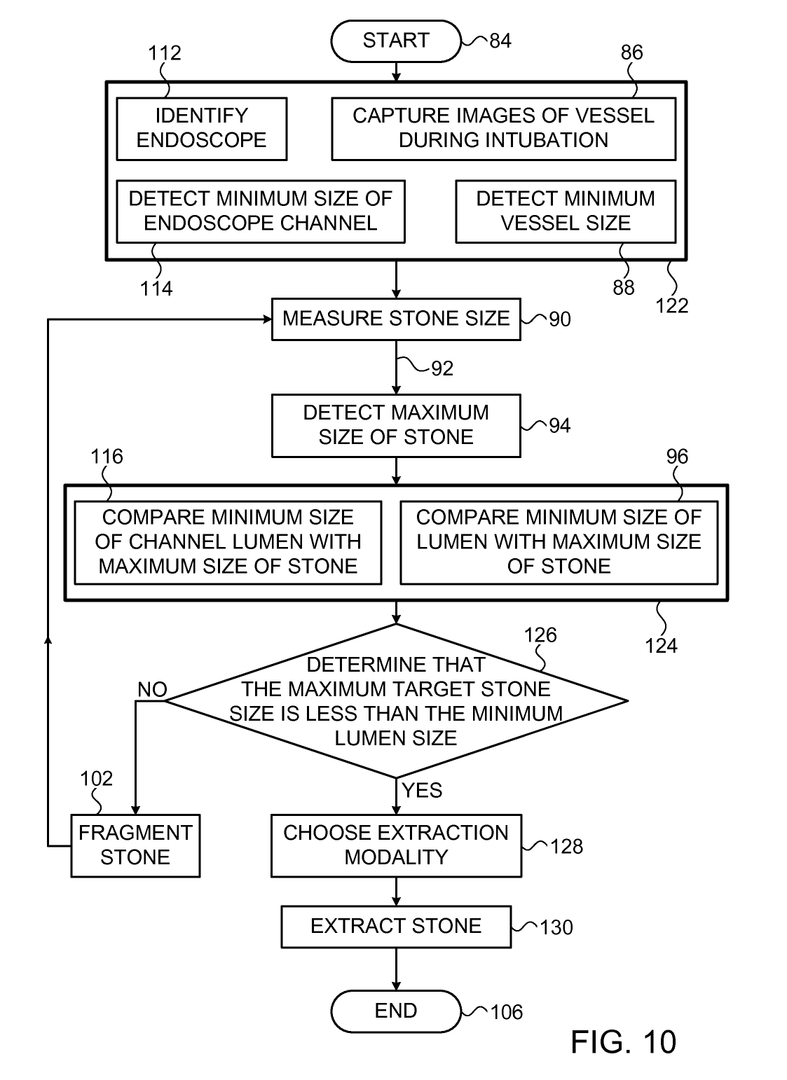

Reference is now made to FIG. 10, which is a flow-chart of a method of method of extraction of a stone, in accordance with an alternate embodiment of the invention. Steps that are performed identically to the methods of FIG. 7 or FIG. 9 are not repeated.

After performing initial step 84, a series of lumen-determining steps represented by block 122 comprises steps 86, 88 (FIG. 7) and steps 112, 114 (FIG. 9). All of the information is memorized.

After performing steps 90, 94 as described above, a comparison is made between the maximum size of the calculus and both minimum sizes of the endoscope channel and the body lumen (e.g., urinary vessel). These comparisons are represented by block 124 and comprise step 96 (FIG. 7) and step 116 (FIG. 9).

Next, at decision step 126, based on the comparisons performed in block 124. it is determined if the calculus is extractable, e.g., the calculus is smaller than either or both the working channel of the endoscope and the body lumen or urinary vessel.

If the determination is affirmative then control proceeds to step 128. The operator determines whether to extract the calculus through the working channel of the endoscope, or through the lumen of the vessel external to the endoscope. After making the determination, the calculus is extracted accordingly in step 130 as described above with respect to step 100 (FIG. 7) or step 120 (FIG. 9). The procedure then terminates at final step 106.

If the determination at decision step 126 is negative then control proceeds to step 102 as described above.

### Third Alternate Embodiment

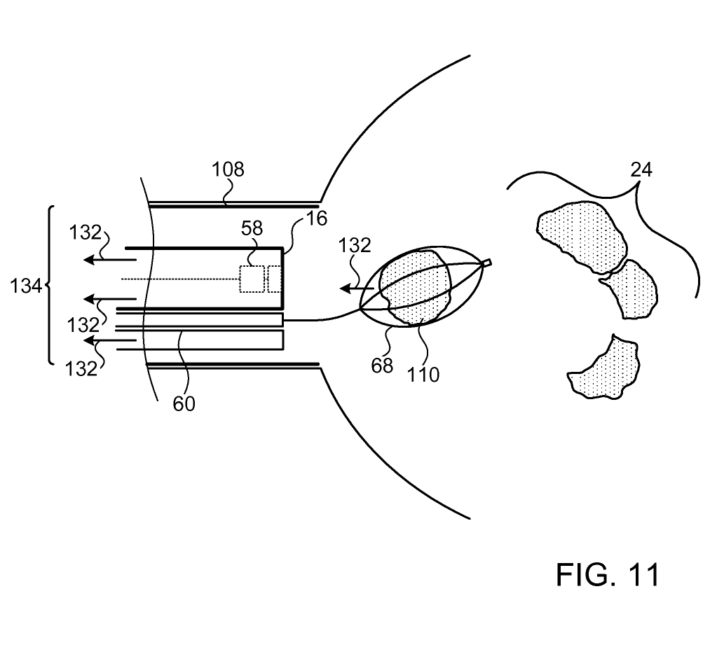

Reference is now made to FIG. 11, which is a schematic diagram illustrating the execution by the endoscope shown in FIG. 1 of a stone retrieval operation in accordance with an alternate embodiment of the invention. This embodiment is similar to the embodiment of FIG. 8, except now the distal end 16 of the endoscope and the basket device 68 are in the process of being retracted through the access sheath 108, (indicated by arrows 132). When the fragment 110 is eventually removed by the basket device 68, the limiting factor is the minimum internal diameter 134 of the access sheath 108. The access sheath 108 includes a dilator, which can expand the lumen of the ureter such that in operation the diameter of the access sheath lumen exceeds the minimum diameter of the body lumen or urinary vessel. The methods of FIG. 9 and FIG. 10 may be modified, mutatis mutandis to use the diameter of the access sheath 108 lumen in place of the value of the minimum size of the body lumen or the channel of the endoscope.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A method for removal of a target stone, the target stone comprising a stone, a stone fragment, or a group of stone fragments, in a body through a lumen comprising the steps of:
detecting a minimum lumen size;
detecting a maximum target stone size of the target stone;
comparing the minimum lumen size and the maximum stone size;
determining that the maximum target stone size is less than the minimum lumen size; and
removing the target stone through the lumen.

2. The method according to claim 1, wherein the detecting steps further comprises:
inserting an imaging device into the body through the lumen of a body;
acquiring at least one first image of the lumen through the imaging device;
acquiring at least one second image of the target stone through the imaging device; and
determining the minimum lumen size and the maximum stone size by processing the images.

3. The method according to claim 1, wherein the detecting steps further comprises:
inserting an imaging device, the imaging device comprising a channel as the lumen to retrieve the target stone and a memory the minimum lumen size is stored; and
comparing the minimum lumen size with the maximum stone size by processing the images.

4. The method according to claim 1, wherein the detecting steps further comprises:
inserting an imaging device through a body lumen, the imaging device comprising a channel lumen to retrieve the target stone and a memory the minimum channel lumen size is stored;
acquiring at least one first image of the body lumen through the imaging device;
acquiring at least one second image of the target stone through the imaging device;
comparing the minimum body lume size with the minimum channel lumen size; and
determining the minimum lumen size and the maximum stone size by processing the images.

5. The method according to claim 1, wherein removing step further comprises:
inserting a retrieval device into the lumen; and
removing the target stone by the retrieval device after the determining step.

6. The method according to claim 1, wherein removing the target stone further comprises:
inserting a lithotripsy device to reduce the maximum size of the target stone; and
removing the target stone, after reduction, by the retrieval device after the determining step.

7. The method according to claim 1, wherein measuring steps further comprises:
acquiring a reference image from a reference object, wherein the reference object having a first known dimension and the lumen or the target stone has a second dimension;
determining a first number of pixels in the reference image that are occupied by the first known dimension;
determining a second number of pixels in the reference image that are occupied by the second dimension; and
calculating a size of the lumen, a size of the target stone, or both by comparing the first number of pixels with the second number of pixels.

8. The method according to claim 7, wherein the reference object comprises a safety guidewire that extends through the vessel.

9. The method according to claim 5, wherein the retrieval device further comprises a reference object to detect at least the size of the body lumen or target stone.

10. The method according to claim 9, wherein the retrieval device further comprises a shaft to be inserted into the lumen, and the reference object is the shaft.

11. The method according to claim 9, wherein the retrieval device further comprises a wire to retrieve the target stone, and the reference object is the wire.
